(19) United States
(12) Patent Application Publication
(10) Pub. No.: US 2006/0173368 A1
(43) Pub. Date: Aug. 3, 2006

(54) Low frequency analysis of cardiac electrical signals for detecting heart diseases, especially the coronary artery diseases

(76) Inventors: Cecilia L. Yu, Hacienda Heights, CA (US); Xiaodong Du, Beijing (CN)

Correspondence Address: Cecilia L. Yu, P.O. Box 93495, City of Industry, CA, 91715-3495, US

(21) Appl. No.: 11/049,166

(22) Filed: Feb. 1, 2005

(51) Int. Cl. A61B 5/04

(52) U.S. Cl. 600/509

(57) ABSTRACT

A method and system for detecting heart diseases, especially the coronary artery diseases, comprises the steps of obtaining twelve (12) lead cardiac electrical signals from a patient, mathematically transforming the time-domain wave signals into twelve (12) frequency-domain amplitude spectra with one for each of the 12 leads, selecting a number of amplitude readings in the low frequency range of 0 Hz to 25 Hz of the frequency amplitude spectrum density curves for analysis, from a reference clinical database of established diagnostic criterion values selecting diagnostic indexes by which the presence of heart disease is judged, determining the pathological values of diagnostic indexes for each lead, comparing the value of each diagnostic index against the value of said index in the database for detection of heart diseases, compiling and visually displaying all 12 amplitude spectrum density curves with their respective diagnostic indexes in one picture for diagnostic reading thereby accomplishing the detection of heart diseases, compiling and visually displaying the value of the diagnostic indexes indicative of coronary health for all 12 leads in one picture for diagnostic reading thereby accomplishing the detection of coronary artery diseases, further analyzing the cardiac electrical signals of lead II and lead V5 to determine a second set of diagnostic indexes, comparing against a reference clinical database of established diagnostic criterion for these indexes, compiling and displaying the results that are indicative of the patient's coronary health of the left ventricular, thereby accomplishing the detection of coronary artery diseases of the left ventricular.

### FIELD OF INVENTION

The present invention related to a method and system whereby the cardio electrical signals are collected by a plurality of detecting electrodes and transformed into frequency-domain spectra to obtain vital information which is then compiled and visually displayed for the detection of the heart diseases, especially the coronary artery diseases.

### DESCRIPTION OF THE PRIOR ART

Coronary artery diseases have been the leading cause of death in the United States and a major concern in the medical field over the years. With the invention of electrocardiogram (ECG) technology more than 100 years ago, physicians have been interpreting the changes in the ECG to detect various heart diseases, including the coronary artery diseases. The advantage of interpreting ECG is this technique is non-invasive, but the major drawback is that it provides less than 50% in accuracy with even less in specificity. In the last twenty years, with the advance in microprocessor, ECG interpretation has been computerized to eliminate the human error, however since the changes in ECG are generally very minor and in some case none, the improvement in accuracy and specificity has been rather limited. There are many other technologies available to the doctors for the detection of heart diseases, such as the nuclear scanning which is non-invasive but expensive to run, catheterization or coronary angiography which is a invasive and expensive procedure. These testing procedures have often been used as a last test to confirm the existence of heart diseases after positive finding in the preliminary testing.

From the technical point of view, an ECG (electrocardiogram) is a compilation and recording of cardiac electrical signal in time sequence. Since ECG composes a number of different electrical currents, it is a complex time-domain signal. When an area of heart muscle is damaged due to lack of blood supply, the characteristic of electrical currents traveling through the heart muscle is affected with change in amplitude and/or direction. Those changes in some cases will show up in an ECG and can be interpreted to diagnose the existence of heart diseases. However, some of these changes are so very minor and cannot be interpreted by a well trained professional. Therefore, ECG still remains as a preliminary screening tool for the doctors because it gives less than satisfactory results in the accuracy and specificity.

In 1965, Fast Fourier Transformation (FFT), a very efficient algorithm, was developed to implement the Discrete Fourier Transformation which was the most straightforward mathematical procedure to transform a time-domain signal into its frequency components. With the invention and advance of computer technology, the fast Fourier transformation of a complex ECG time-domain signal into its unique frequency components can be accomplished in a second. With the FFT, a tremendous amount of research work has been done in the recent years in the analysis of the frequency-domain components of a non-invasive ECG for the detection of heart disease. For instance, Chamoun's patent (U.S. Pat. No. 5,020,540) described a method and system of choosing and extracting an arrhythmia-free QRST complex from a time-domain ECG as a template and analyzing its frequency components in a very high frequency range (150-250 Hz) to detect various types of heart diseases. The shortcoming in this approach are two folds, one is Chamoun's per-determination to use only an arrhythmia-free QRST complex for frequency analysis which artificially excludes a group of patients from testing. The second one is Chamoun's only uses the high frequency components in the range of 150 to 250 Hz for the analysis when a major portion of the cardiac electrical frequency components after FFT transformation are in the 0 to 50 Hz frequency range. Chamoun's overlook of the low frequency components from 0 Hz to 25 Hz of an ECG complex leaves a big gap in the research spectrum. The present invention without predetermination of which segment of ECG signal should be use looks at the entire cardiac electrical signals in their low frequency range of 0 to 25 Hz which a treasury of useful information is located.

At the time of Chomoun's patent, Shen's patent (U.S. Pat. No. 5,029,082) revealed an apparatus for detecting and processing electrocardiogram (ECG) signals for two selected leads in their frequency domain. Later, Feng in his two patents (U.S. Pat. Nos. 5,509,425 and 5,649,544) carried out more research work using the same ECG signal from two selected leads, Lead II and Lead V5, as in Shen's patent for frequency analysis. Both Feng's patents describe a method to mathematically determine a plurality of functions and a set of indices for each function for diagnosing a cardiac condition and warning of heart attack of a patient. However, there are many shortcomings in Feng's approach. Feng's patent uses ECG signal collected form two selected leads, II and V5, for frequency analysis and fails to give due consideration of the useful information from other cardiac electrical signals collected by other ten ECG leads and thus unnecessarily forfeited the benefit from their analysis. The other concern in Feng's patents is neither of Feng's two patents has ever established any relationship between the six functions and 73 indices identified in the patents, and how these indices can be used for the detection of heart diseases. Presumably each of those indices is to be used to detect heart disease, but Feng's patents do no provide any explanation or direction on how to use those indices.

After Feng's patents, frequency analysis of ECG signals from all 12 leads was described later in Fang's patents (U.S. Pat. Nos. 6,148,228 and 6,638,232) entitled system and method for detecting and locating heart diseases. A base value is obtained by multiplying a patient's heart beats per second by a scaling quantity of 5, and then comparing the area of a power spectrum from 0 Hz to the base value over the area from said base value to infinite to get an area ratio, and then using the area ratio to establish an evaluation standard indicative of coronary artery diseases. Although Fang's patents did not mention that the technology involved is of low frequency analysis, the area calculated in Fang's patents is from 0 Hz to the base value of 5 Hz and fro the base value of 5 Hz to infinite in frequency. They nevertheless cover the low frequency range of 0 Hz to 25 Hz. Fang's patents also use the cardiac electrical signals of all 12 leads for his frequency analysis. The shortcomings in Fang's patents are that first the scope of the work is limited to the use of a base value and area ratio to locate the coronary artery diseases and secondly the failure to use the information from the frequency components of all 12 leads to achieve a more systematic and efficient approach to detect the heart diseases.

The present invention provides a method and system for a visual systematic approach in the detection of the heart diseases. Seeing is a thousand words. This invention is the first to provide a visual reading of spectrum supported by diagnostic indexes. It examines the frequency components in the low frequency range from 0 Hz to 25 Hz for cardiac electrical signals from all 12 leads, determines their respective diagnostic indexes, compiles all the information in three unique visual displays. The first visual display shows 12 frequency spectra, one for each lead, with their respective indexes for diagnosis of arrhythmia, hypertrophy, myocardium injuries and coronary artery diseases from ischemia to myocardial infarction. The second visual display shows the value of the diagnostic indexes indicative of coronary health for all 12 leads in one picture for diagnostic reading thereby accomplishing the detection of coronary artery diseases. The third and final visual display is to check the health of the left ventricular for any serious diseases that may cause heart attack.

### SUMMARY OF THE INVENTION

An object of the present invention is to provide a method and system of low frequency analysis of the cardiac electrical signals of all 12 leads for the detection of heart diseases, especially the coronary artery diseases.

Another object of the present invention is to provide a method and system of compiling and displaying the diagnostic information of all 12 lead for visual reading and detecting the presence of heart diseases.

Another object of the present invention is to provide a method and system of compiling and displaying the diagnostic information for coronary health of the heart for visual reading and detecting the presence and location of coronary artery disease.

Yet another object of the present invention is to provide a method and system of compiling and displaying the diagnostic information of low frequency analysis of two leads, II and V5, for visual reading and detecting patient's coronary health of the left ventricular, thereby accomplishing the detection of coronary artery diseases in the left ventricular, especially the presence of myocardium infarction, a serious form of coronary artery diseases.

### BRIEF DESCRIPTION OF THE DRAWINGS

FIG. 1 is a schematic diagram of the apparatus components of the system used in the present invention;

FIG. 2 is an overview flow chart of the operation of the system used in the present invention;

FIG. 3, is a picture of a frequency amplitude spectrum density curve along with the diagnostic indexes generated by the method and system of the present invention for one of the 12 leads

FIG. 4 is the graphical representation of one of the diagnostic outputs provided by the method and process of the present invention showing the visual display of 12 individual frequency amplitude spectrum density curves with one for each lead plus respective diagnostic indexes generated in connection with the established clinical diagnostic criterions;

FIG. 5 is the graphical representation of one of the diagnostic outputs provided by the method and system of the present invention showing the visual display of 12 individual coronary artery disease detection columns with the diagnostic line generated in connection with the established clinical diagnostic criterions.

FIG. 6 is the graphical representation of one of the diagnostic outputs provided by the method and system of the present invention showing the visual display of 2 individual frequency amplitude spectrum density curves, one for lead II and the other for lead V5, and the graphs for signal analysis for these two leads plus the diagnostic indexes generated in connection with the established clinical diagnostic criterions.

### DESCRIPTION OF THE PREFERRED EMBODIMENTS

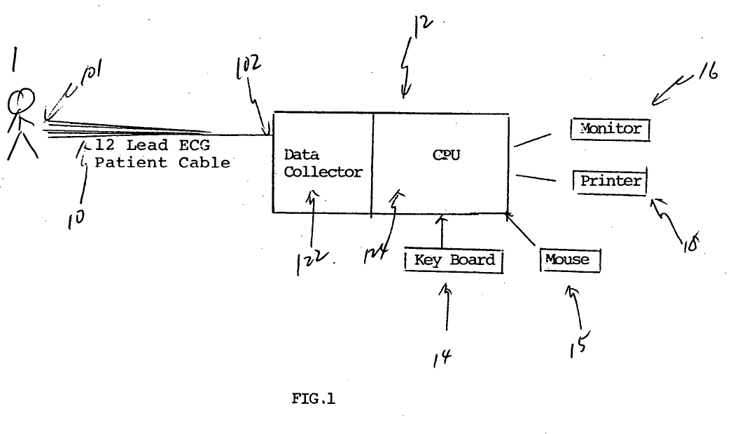

Referring to FIG. 1, the apparatus used in the present invention consists of a conventional 12 lead electrocardiograph (ECG) patient cable 10 having a set of ten surface electrodes (collectively 101) attached to the surface of a test subject 1 at the prescribed positions, and the other end 102 of the 12 lead ECG patient cable 10 connected to a computer 12. This computer 12 consists of a data collector 122 and a CPU unit 124. The data collector 122 has a serial port to connect the 12 lead ECG patient cable 10, a A/D converter, a amplifier, filters, and an electrical isolator. The CPU 124 is a micro processor to process the input information. The computer 12 is connected to a monitor 16 and a printer 18. The monitor 18 is for visual display and the printer 16 is to print out the output information on commend by the computer 12 operated by a keyboard 14 and a mouse 15 connected to the computer 12 for the operation.

The cardiac electrical signals 20 are collected by the electrodes 101 of a 12 lead ECG patient cable 10, transmitted to a data collector 122. After the signals 20 have been digitized by an A/D converter, amplified by an amplifier, filtered off the noise by a filters, and transmitted through the electrical isolator to a CPU 124. The CPU 124 has been pre-installed with a proprietary software and a proprietary database of clinical studies to perform the data analysis. Once the cardiac electrical signals 20 are transmitted into the CPU 124, the CPU 124 will analyze the information and present the information commended by a operator using a keyboard 14 and mouse 15.

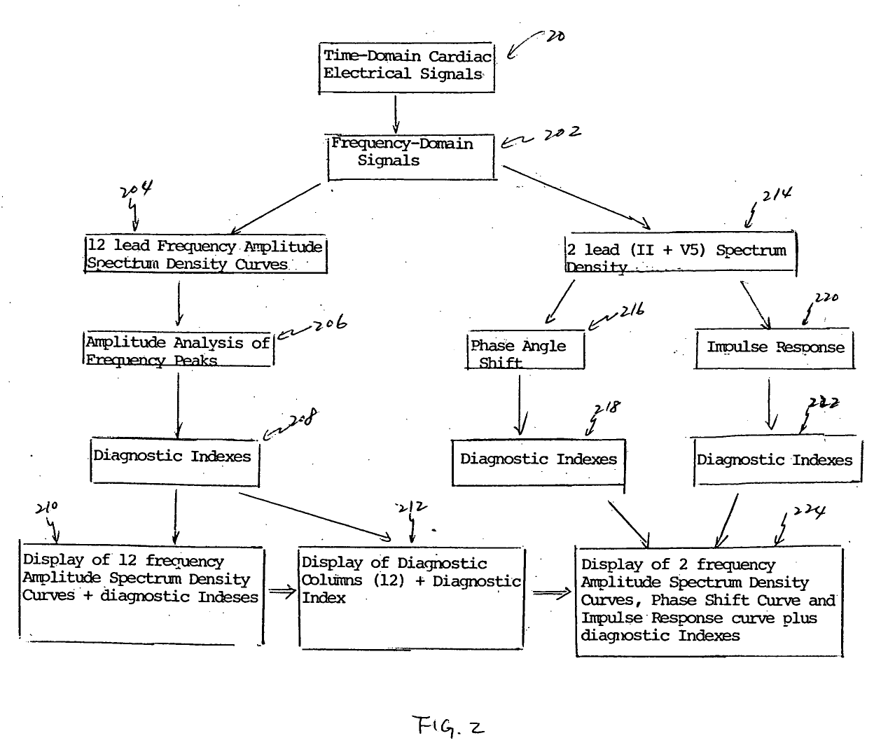

Referring to FIG. 2, an overview of the flow chart showing the operation of the system of the present invention. The cardiac electrical signals 20 collected by each of a 12 lead ECG patient cable 10 of a tested subject 1 are first mathematically transformed from time-domain into their respective frequency data 202 by means of Fast Fourier Transformation equations. These frequency data 202 are plotted into 12 separate and individual frequency amplitude spectrum density curves 204. From each of the frequency amplitude spectrum density curves, a number of frequency peaks are selected and their pathological value calculated and labeled with an alphabetic letter as the diagnostic indexes 206. Comparing the pathological values of these diagnostic indexes against the pathological values that have been established clinically and store in the database to determine a positive (“+”) sign or a negative (“−”) sign for those diagnostic indexes 208. Once the sign for each diagnostic index has been identified, each of the 12 frequency amplitude spectrum density curves together with diagnostic indexes plus each of the sign are compiled together and displayed 210 either with a monitor and printed out by a printer. If there are more than one positive (“+”) index shown for any one of the 12 the frequency amplitude spectrum density curves 41-52, the next recommended step is to obtain the second display to find out whether there is coronary artery diseases 212.

From diagnostic indexes 208, one index is selected. Takes out the already calculated pathological value for said index for each of the 12 leads and plots against said respective lead to development a graphical display of a plurality of multi-colors diagnostic columns for the detection and location of coronary artery diseases 212.

The third step is to analyze the frequency components of the cardiac electrical signals from lead II and lead V5 214. The reason of selecting these two leads is because the cardiac electrical signals detected by these two lead travel through the frontal left ventricular area of a heart where coronary diseases would cause more serious consequences than that of other part of the heart. Adopting the two fundamental methods used in the digital signal processing to study the transmission function of a device, transfer function in phase angle shift and impulse response, to analyze the frequency components in the frequency range form 0 Hz to 25 Hz of the cardiac electrical signals from these two leads 216. Using the cardiac electrical signal of lead V5 as the input signal and lead II as the output signal. Pathological values for the phase angle shift of the transfer function of these two leads are calculated, compared against said value in the clinically established database to determine a diagnostic index has a positive (“+”) sign or a negative (“−”) sign 218. The degree in phase angle shift is plotted against the frequency from 0 Hz to 25 Hz to get a phase shift curve. Compile the diagnostic index and phase shift curve in one picture 224 which can be displayed on a monitor 16 or printed out by a printer 18 for visual diagnosis.

The degree in phase angle shift is used to measured the conductivity function of a heart, particularly the frontal area of the left ventricular. Further diagnosis for the nature and scope of coronary artery disease analysis of left ventricular is carried out with the use of mathematical approach of impulse response in the digital signal processing 214. In the present invention, pathological values for the impulse response between these two leads are calculated, and diagnostic indexes are selected and labeled by alphabetic letters. A positive (“+”) sign or a negative (“−”) sign is determined for each of the diagnostic indexes comparing against said values in the clinically established database 222. The impulse response co-efficiency verses time in milli-second is plotted to give an impulse response curve. Putting together the frequency amplitude spectrum density curves for lead II and lead V5, phase angle shift curve, impulse response curve and their respective diagnostic indexes, a two lead visual diagnostic analysis is compiled in one picture 224 which can be displayed on a monitor 16 or printed out by a printer 18.

It is recommended that a diagnostic procedure starts with the visual analysis of 12 lead frequency amplitude spectrum density curves 210. If the diagnostic indexes indicate possible presence of coronary artery diseases, the next step is to go to the visual display of multiple diagnostic columns 212 to find out which area of heart has coronary artery disease. Once the existence of the coronary artery disease is confirmed, then go to the two lead visual diagnostic analysis 224 to detect any heart diseases in the left ventricular.

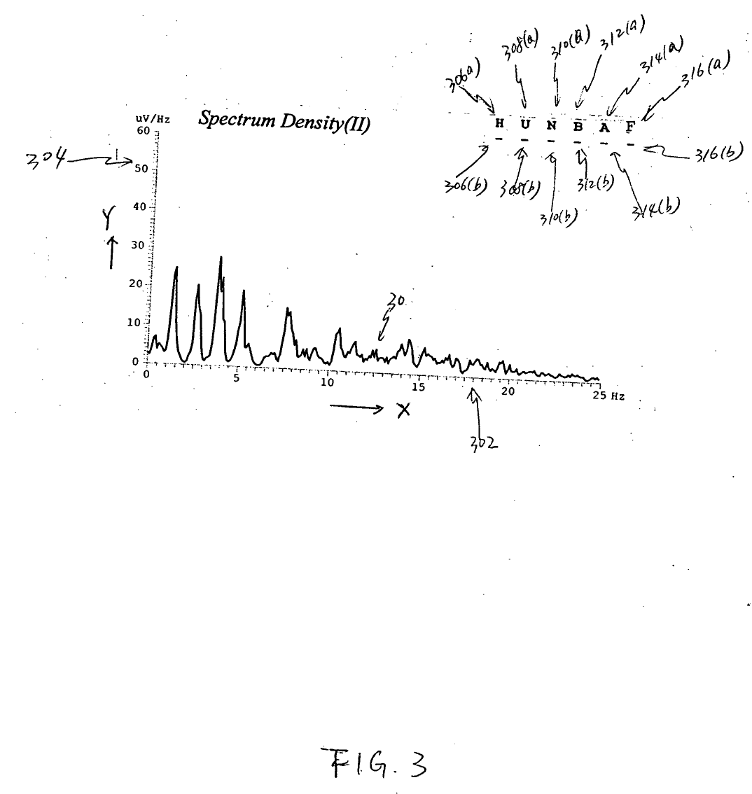

Referring FIG. 3, a picture of a frequency amplitude spectrum density curve along 30 with the diagnostic indexes generated by the method and system of the present invention for one of the 12 leads. The frequency amplitude spectrum density curve 30 is a result of plotting the value of frequency (Hz) as the horizontal (X) axis 302 and the value of amplitude (uV) as the vertical (Y) axis 304. H 306(a), U 308(a), N 310(a), B 312(a), A 314(a), F 316(a) are the diagnostic indexes in alphabetic letters, and 309(b), 308(b), 310(b), 312(b), 314(b), 316(b) are sign of positive (“+”) or negative (“−”) for each of the six diagnostic indexes after compared against the established diagnostic criterions in the database.

The letter index H 306(a) is a diagnostic index established by analyzing a database of clinical diagnostic criterions for detection of insufficient myocardial power in the myocardium caused by lack of blood supply. The pathological value for index H 306(a) is calculated by comparing the amplitude value of the first and second peak in the spectrum density curve. If the amplitude ratio of the second peak over the first peaks is more than 0.75, the letter H 306(a) index has a sign 306(b) of positive (“+”) which indicates that the area of heart where the lead points to does not have sufficient myocardial power due to insufficient blood supply to that area. This is the beginning stage of developing coronary artery diseases.

The letter index U 308(a) is a diagnostic index established by analyzing a database of clinical diagnostic criterions for detection of arrhythmia. The pathological value for index U 308(a) is calculated by measuring the distance between one frequency peak and its adjacent frequency peak in a frequency amplitude spectrum density curve and comparing all the peak-peak distance for the entire frequency range from 0 Hz to 25 Hz. If there is a discrepance in the distance among the adjacent two peaks which means some distance is larger or smaller than the others, the letter index U 308(a) has a sign 308(b) of positive (“+”) which indicates that the tested subject has arrhythmia.

The letter index N 310(a) is a diagnostic index established by analyzing database of clinical diagnostic criterions for detection of injuries in myocardium. The pathological value of index N 310(a) is calculated by measuring the amplitude value of the first peak in a frequency amplitude spectrum density curve. If at the frequency point where the first peak should be shows no amplitude value or less than 3 uV in amplitude, the letter index N 310(a) has a sign 310(b) of positive (“+”) which indicates the presence of myocardial injuries.

The letter index B 312(a) is another diagnostic index established by analyzing the database of clinical diagnostic criterions for detection of hypertrophy. The pathological value of index B 312(a) is calculated by measuring the amplitude value of the first and second peaks in the frequency amplitude spectrum density curve. When the mathematical sum in uV from the first peak and second peak is over 80 uV, the index B 312(a) has a sign 312(b) of positive (“+”) which indicates the presence of hypertrophy.

The letter index A 314(a) is another diagnostic index found to have diagnostic significance based on the database of clinical diagnostic criterions for detection of early stage of ischemia. The pathological value is calculated by comparing the amplitude values of the second peak against the amplitude value of the first peak. If one of the pathological value is over 1.00, the index A 314(a) has a sign 314(b) of positive (“+”) which indicates the presence of ischemia.

The letter index F 316(a) is another diagnostic index that has been clinically established for the detection of use of myocardial compensation which generally takes one or two years to develop after having ischemia. The pathological value is calculated by comparing the amplitude values of any one peak from the fifth peak to the thirtieth peak against the amplitude value of the first peak. If one of the pathological value is over 0.75, the index F 316(a) has a sign 316(b) positive (“+”) which indicates the heart has used myocardial compensation to carry its pumping function as a consequence of prolong and advanced ischemia, or myocardial infarction.

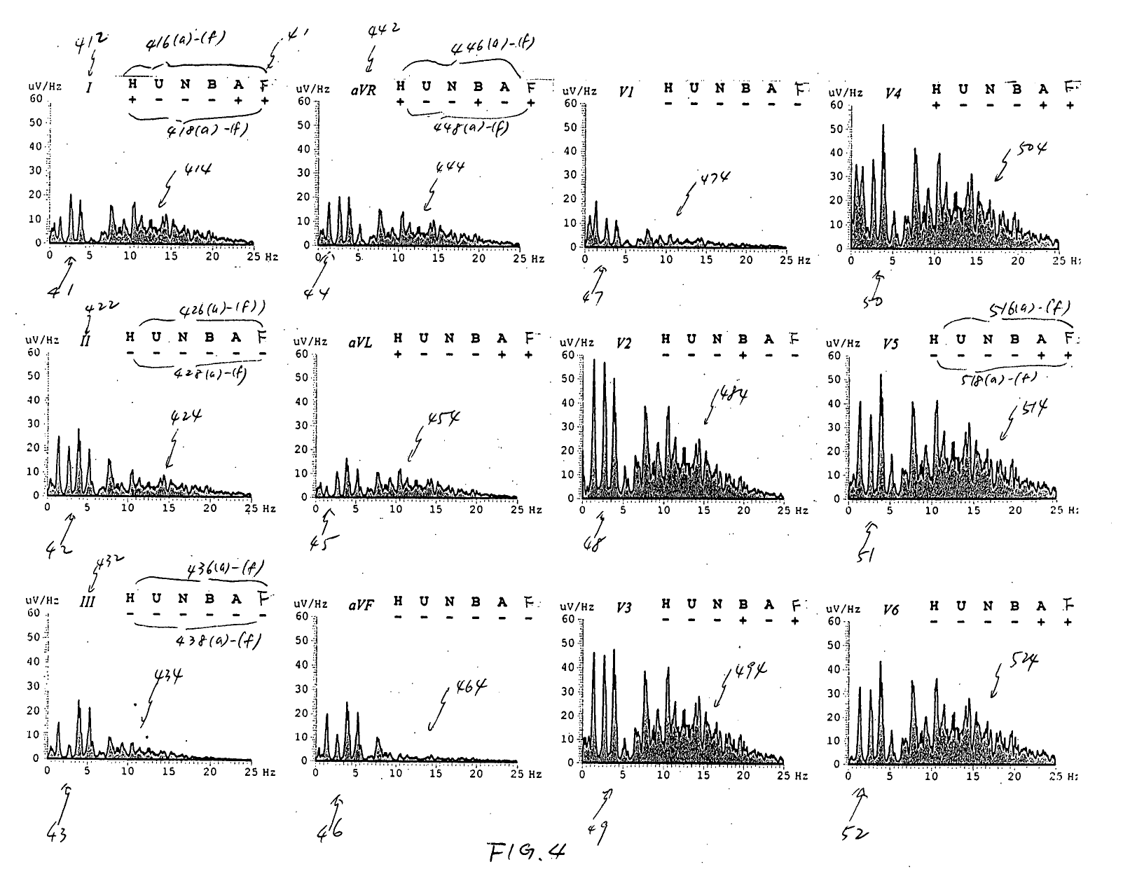

Referring to FIG. 4, a graphical representation 40 of the first of three diagnostic outputs provided by the method and process of the present invention showing a visual display of 12 individual frequency amplitude spectrum density curves with one for each of the 12 leads along with their respective diagnostic indexes generated after comparing against the established clinical diagnostic criterions. There are 12 frequency amplitude spectrum density curves 41-52, one for each of the 12 leads. Starting from the one on the left hand upper corner 41, there is an alphabetic letter I 412 specifying that this spectrum density curve 414 is the spectrum density curve for lead I, the six alphabetic letters to the right of the letter I 412 are the same six diagnostic indexes as identified and explained in FIG. 3. They are H 416(a), U 416(b), N 416\textcircled{C}), B 416(d), A 416(e) and F 426(f) with their respective positive (“+”) sign or negative (“−”) sign 418(a)-(f) directly under said each of the alphabetic letters.

This graphical representation 40 shows 12 separate and individual spectrum density curves places in three rows with four in one row. The first and top row 40(a) shows the spectrum density curves for lead I 41, lead aVR 44, lead V1 47 and lead V4 50. The second and middle row 40(b) has also four spectrum density curves for lead II 42, lead aVL 45, lead V2 49 and lead V5 51. The last and bottom row shows four spectrum density curves for lead III 43, lead aVF 46, lead V3 49 and lead V6 52. Each curve also has the diagnostic indexes in alphabetic letters (a)-(f) and their respective positive (“+”) or negative (“−”) signs (a)-(f).

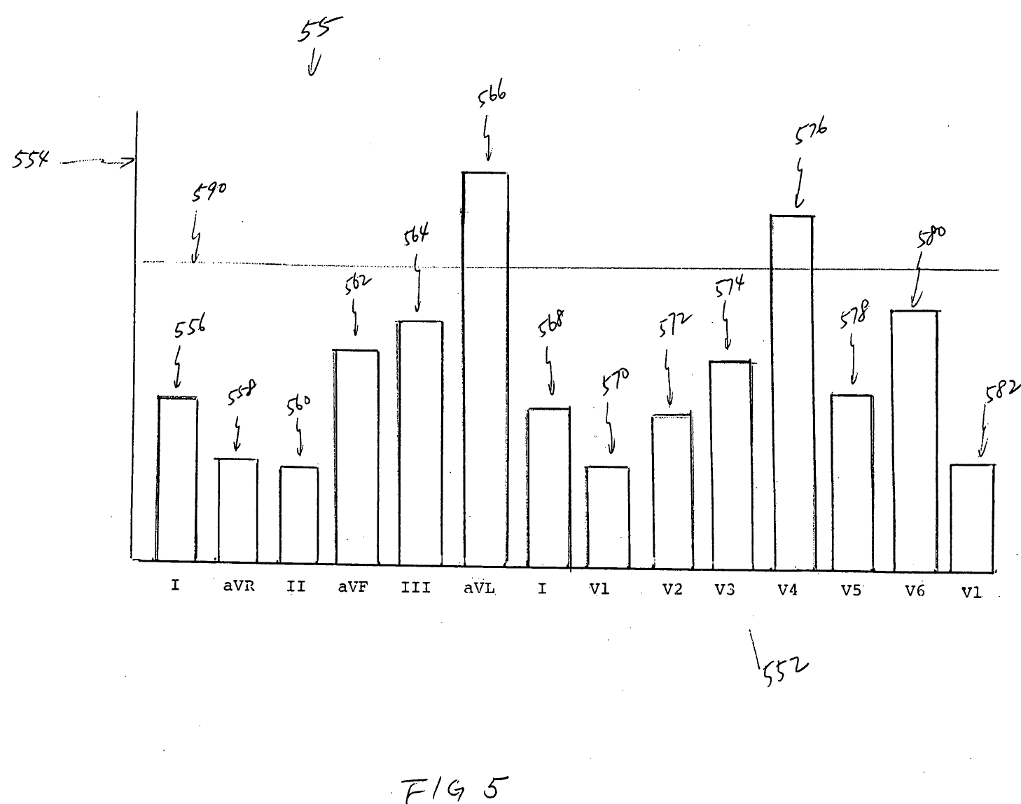

Referring to FIG. 5, a graphical representation 55 of the second of three diagnostic outputs provided by the method and system of the present invention showing the visual display of fourteen individual coronary artery disease detection columns with a diagnostic line generated by the method and system of the present invention in connection with the established clinical diagnostic criterions. The displaying representation 55 is a plot of pathological value in uV and identification of each lead. The pathological value in uV is for the vertical (Y) axis 554 and the identification of each lead is the horizontal (X) axis 552. Using the pathological value of a selected diagnostic index for each of the 12 leads and plot against their respective individual lead in two lead groups in the order of I 556, aVR 558, II 560, aVF 562, III 564, aVL 566, I 568 for the limb group, and V1 570, V2 572, V3 574, V4 576, V5 578, V6 580, V1 582 for the chest group, to develop a graph with a total of 14 multi-colors diagnostic columns 556-582. There is a horizontal line called “diagnostic line” 590 starting at a point of Y axis 554 where the pathological value for which the positive (“+”) and negative (“−”) index is determined, and traveling parallel all the way to the end of the X axis 552. When a diagnostic column for one lead passes over the diagnostic line 590, it indicates an abnormal condition at the area where said lead points to. The color of each column is blue at the base line 552 and changes gradually to green and then yellow as the pathological value for said lead increases and the column moves upward until it reaches the diagnostic line 590. Once the column goes over the diagnostic line 590, the color changes from orange and then red as the pathological value increases more. To detect the general location of coronary artery diseases, we observe how many columns go over the diagnostic line 590. When there are three consecutive columns in one group, limb group and/or chest group, going over the diagnostic line 590 with top layer of the color being orange or red, it indicates the presence of coronary artery disease at the area where the lead having the tallest column points to.

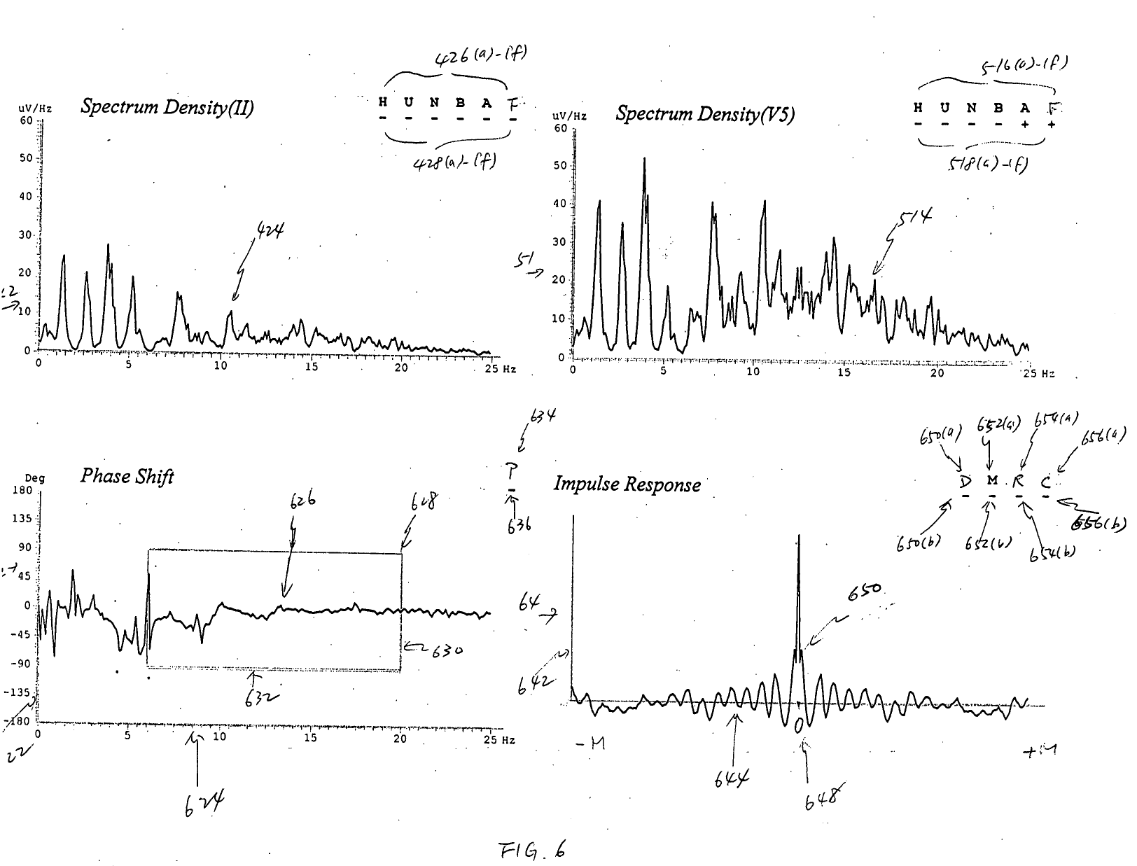

Referring FIG. 6, a graphical representation of the last of three diagnostic outputs provided by the method and system of the present invention showing the visual display of two individual frequency amplitude spectrum density curves, one for lead II and the other for lead V5, and two figures, phase shift and impulse response, of signal analysis of same two leads plus diagnostic indexes generated after comparing with the established clinical diagnostic criterions.

This display representation consists of four graphs, two in the first and top row and two in the second and bottom row. The two graphs in the top row are the frequency amplitude spectrum density curves together with six diagnostic indexes and their respective positive or negative sign for lead II 42 and lead V5 51. The two graphs in the bottom row are the phase shift curve 62 for the transfer function in phase angle shift and impulse response curve 64 for the same two leads.

Phase shift is the angle shift between the input current and output current. In method and system of the present invention, the cardiac electrical current of lead V5 is used as the input current and the cardiac electrical current of lead II as the output current. Applying the mathematic equations for the transfer function in phase angle shift in digital signal processing to calculate the degree of phase shift between these two signals at every 0.0025 Hz frequency increment for the entire frequency range from 0 Hz to 25 Hz.

For the present invention, a phase shift curve 62 is a plot of degree of phase shift angle 622 from −180 degree to +180 degree as the vertical (Y) axis, and the frequency 624 from 0 Hz to 25 Hz as the horizontal (X) axis. The angle in phase shift at every frequency from 0 to 25 Hz is calculated and plotted to give a phase shift curve 626. There is a rectangular area 628 outlined from the phase shift angle of −90 degree to +90 degree of the Y axis 630 and 6 Hz to 20 Hz of the X axis 632 where diagnosis is carried out. The pathological value is calculated by measuring every phase shift angle against the absolute 90 degree from 6 to 20 Hz. If the calculated value is larger than 1.00, then the diagnostic index P 634 had a sign 636 of positive (“+”) which indicates abnormality in the myocardial conduction function. In addition, this diagnosis can be accomplished by observing how the phase shift curve travels within this diagnostic rectangular area 628. When the phase shift curve 626 at any frequency point between 6 Hz to 20 Hz 632 travels out side of the rectangular area 628, it indicates abnormality in myocardial conduction function.

In digital signal processing, when a system is stimulated with an electrical impulse, the output response can also be mathematically calculated by the inversed Fourier transformation of the transfer function in amplitude. In the method and system of the present invention, the cardiac electrical current of lead V5 is treated as the stimulating input current and the cardiac electrical current of lead II is treated as the excited output current. The relative impulse response of cardiac electrical current of lead V5 when stimulated by the cardiac electrical current of lead II is mathematically calculated for every frequency point in 0.0025 Hz frequency increment from 0 Hz to 25 Hz frequency range. The value for impulse response relativity 642 as the vertical (Y) axis is plotted against certain time intervals 644 from time −M to 0 to +M where M is a non-zero integral value in milli-second to get a impulse response cure curve 646.

Based on the clinically established criterions for a healthy heart, for a impulse response graph 64, there is only one narrow and sharp peak standing above the base line of the impulse response curve 646 at zero (0) point 648 and this peak is identified as the main peak 650. The pathological value is calculated by detecting the existence of any peak other than the main peak 650 above or below the impulse response base line from time −M to +M. Four diagnostic indexes were developed from the database D 650(a), M 652(a), R 654(a), and C 656(a) and their respective sign of 650(b)-656(b) of positive (“+”) or negative (“−”). When the main peak 650 is at an inversed position from the X axis at zero (0) point 648, the diagnostic index D 650(a) has a positive (“+”) sign 650(b) which indicates coronary artery diseases in the left ventricular. When there are multiple peaks in place of the main peak 650, the diagnostic index M 652(a) has a sign 652(b) of positive (“+”) which indicates poor conduction function in the left ventricular. When there is one peak on each side with a distance from the main peak 650, the diagnostic index R 654(a) has a sign 654(b) of positive (“+”) which indicates advanced ischemia with possible myocardial infarction in the left ventricular. When there is multiple peaks on either side of the main peak 650, but not replacing the main peak 650, the diagnostic index C 656(a) has a sign 656(b) of positive (“+”) which indicates latent arrhythmia.

In the method and system of the present invention, diagnosis of the left ventricular can be further accomplished by visually observing the impulse response curve along with these four indexes 650(a)-656(a) and their respective sign 650(b)-656(b) of positive (“+”) or negative (“−”) for each index for coronary artery diseases, poor conduction function, ischemia with possible myocardial infarction and latent arrhythmia.

What is claimed is:

1. A method for non-invasively detecting heart diseases, especially for detecting and locating the coronary artery diseases, comprising the steps of:
obtaining time-domain cardiac electrical signals from a patient using a conventional electrocardiograph (ECG) cable with ten surface electrodes;
mathematically transforming the time-domain cardiac electrical signals into frequency-domain components;
selecting a number of frequency peaks in low frequency range from 0 Hz to 25 Hz for signal processing;
generating pathological values from selected frequencies for each lead;
generating pathological values from frequency signals of selected two leads;
comparing said pathological values to reference pathological values stored in the database of clinical studies to determine a number of diagnostic indexes; and
compiling and displaying frequency-domain components, pathological values and diagnostic indexes for detecting and locating heart diseases. artery diseases.

2. The method of claim 1 wherein said time-domain cardiac electrical signals are signals from all 12 leads.

3. The method of claim 1 wherein said mathematically transforming time-domain cardiac electrical signals into frequency-domain components uses Fast Fourier Transformation equations;

4. The method of claim 1 wherein the transformation from time-domain signals into frequency domain components is done concurrently for each of 12 leads.

5. The method of claim 1 wherein said frequency-domain components and corresponding amplitude for each lead of said 12 leads is recorded and plotted to give 12 individual frequency spectrum density curves.

6. The method of claim 1 wherein said number of selected frequency peaks in low frequency range from 0 Hz to 25 Hz for signal processing is 1-30.

7. The method of claim 1 comprising steps of using amplitude values of selected peaks from frequency spectrum density curves to generate pathological values;

8. The method of claim 1 wherein said pathological values are generated for each of said 12 leads.

9. The method of claim 1 further comprising steps of determining the positive (“+”) or negative (“−”) sign for each of said diagnostic indexes comparing said pathological values to reference pathological values in said database that have been diagnostic criterions established clinically for each of 12 leads.

10. The method of claim 1 wherein said number of indexes is 1-10.

11. The method of claim 1 wherein each index is identified by an alphabetic letter.

12. The method of claim 9 wherein a positive (“+”) diagnostic index indicates abnormal condition and a negative (“−”) index indicates normal condition.

13. The method of claim 1 wherein said 12 frequency spectrum density curves with said respective diagnostic indexes in alphabetic letters and “+” or “−” sign are compiled and displayed in one picture to give a visual diagnostic reading of said patient.

14. The method of claim 13 wherein said visual diagnostic reading of heart is to analyze the presence of arrhythmia in said patient.

15. The method of claim 13 wherein said visual diagnostic reading of heart is to analyze the presence of hypertrophy in said patient.

16. The method of claim 13 wherein said visual diagnostic reading of heart is to analyze presence of ischemia in said patient.

17. The method of claim 13 wherein said visual diagnostic reading of heart is to analyze presence of myocardium injuries in said patient.

18. The method of claim 1 further comprising steps of selecting diagnostic indexes from each lead and compiling said diagnostic indexes from all 12 leads in one picture to give a visual display for diagnostic reading for presence and location of coronary artery diseases.

19. The method of claim 18 wherein said number of diagnostic index selected is 1-3.

20. The method of claim 18 comprising steps of using pathological value of selected diagnostic index to generate diagnostic column for visual diagnostic display.

21. The method of claim 20 wherein said diagnostic column is generated for each of the 12 leads.

22. The method of claim 20 wherein said visual diagnostic display consists of 14 columns with seven columns in group, one for limb leads and the other one for chest leads.

23. The method of claim 22 wherein said limb leads are arranged in the order of I, aVR, II, aVF, III, aVL, I.

24. The method claim 22 wherein said chest leads are arranged in the order of V1, V2, V3, V4, V5, V6, V1.

25. The method of claim 20 wherein said column is colored with a scale of multiple colors in the shade of blue, green, yellow, orange and red.

26. The method of claim 20 wherein said visual diagnostic display has a diagnostic line to separate the normal and abnormal conditions.

27. The method of claim 20 wherein diagnosis is normal when said column is below said diagnostic line and the color is in the shade of blue, green and yellow.

28. The method of claim 20 wherein diagnosis is abnormal when said column is above said diagnostic lien and the color is in the shade of orange and red.

29. The method of claim 18 wherein coronary artery disease is ischemia.

30. The method of claim 18 wherein said visual diagnostic reading for presence of coronary artery diseases provide location of said coronary artery diseases in said patient.

31. The method of claim 18 wherein when a plurality of consecutive columns in said group are over said diagnostic line, said location is identified by lead having the tallest column.

32. The method of claim 1 wherein said selected two lead to generate pathological value for a number of diagnostic indexes are lead II and lead V5.

33. The method of claim 32 wherein said pathological values are generated using the mathematic equations for transfer function in phase angle shift in digital signal processing.

34. The method of claim 32 further comprising the steps of determining positive (“+”) or negative (“−”) sign for said diagnostic indexes comparing said generated pathological values to reference pathological values in said database that have been diagnostic criterions established clinically for said two leads.

35. The method of claim 32 wherein said number of indexes is 1-4.

36. The method of claim 32 wherein each index is identified by an alphabetic letter.

37. The method of claim 32 wherein a positive (“+”) diagnostic index indicates abnormal condition and a negative (“−”) index indicates normal condition.

38. The method of claim 33 wherein said pathological values for transfer function in phase angle shift is calculated using said signals in frequency range of 0 Hz to 25 Hz.

39. The method of claim 33 wherein said pathological values for transfer function in phase angle shift is plotted against frequency to give a phase shift curve.

40. The method of claim 32 wherein said diagnostic indexes in alphabetic letters and their respective “+” or “−” sign are compiled and displayed together with said phase shift curve in one picture to give visual diagnostic reading of area where lead II and lead V5 indicate.

41. The method of claim 32 wherein said diagnostic reading is to analyze performance of conductivity function of heart in said patient.

42. The method of claim 32 herein said generated pathological values are determined from said calculated values using mathematic equations for impulse response in digital signal processing.

43. The method of claim 42 further comprising steps of determining positive (“+”) or negative (“−”) sign for diagnostic indexes comparing said generated pathological values to reference pathological values in said database that have been diagnostic criterions established clinically for said two leads.

44. The method of claim 43 wherein said number of indexes is 2-7.

45. The method of claim 44 wherein each index is identified by an alphabetic letter.

46. The method of claim 43 wherein a positive (“+”) diagnostic index indicates abnormal condition and a negative (“−”) index indicates normal condition.

47. The method of claim 42 wherein said values of impulse response are calculated for signals in frequency range of 0 Hz to 25 Hz.

48. The method of claim 42 wherein said values calculated are plotted against time to obtain an impulse response curve.

49. The method of claim 1 wherein said phase shift curve, impulse response curve, diagnostic indexes in alphabetic letters and their respective “+” or “−” sign together are compiled and displayed in one picture to give visual diagnostic reading of area where lead II and lead V5 point to.

50. The method of claim 1 wherein said visual diagnostic reading of the area where lead II and lead V5 point to is to evaluate the degree of ischemia in left ventricular of a heart in said patient.

51. A system for non-invasively detecting heart diseases, especially for detecting and locating the coronary artery diseases, comprising in combination:
means for obtaining 12 lead time-domain cardiac electrical signals from a patient using a conventional electrocardiograph (ECG) cable with ten surface electrodes;
means for mathematically transforming time-domain cardiac electrical signals into frequency-domain data;
means for selecting a number of frequency peaks in low frequency range from 0 Hz to 25 Hz for signal processing;
means for generating pathological values from selected frequencies;
means for generating pathological values from frequency signals of selected two leads;
means for comparing said pathological values to reference pathological values stored in the database of clinical studies to determine diagnostic indexes; and
means for compiling and displaying diagnostic indexes for detecting and locating heart diseases artery diseases.

52. The system of claim 51 wherein the means for mathematically transforming the time-domain cardiac electrical signals into frequency-domain signals comprising the use of Fast Fourier Transformation equations.

53. The system of claim 51 wherein said selected two leads are lead II and lead V5.

54. The system of claim 51 wherein said pathological values are the frequency spectrum density values.

55. The system of claim 51 wherein said pathological values are the transfer function in phase angle shift values.

56. The system of claim 51 wherein said pathological values are the impulse response values.
